(19) United States
(12) Patent Application Publication
(10) Pub. No.: US 2007/0155022 A1
(43) Pub. Date: Jul. 5, 2007

(54) Degenerate binding detection and protein identification using Raman spectroscopy nanoparticle labels

(76) Inventors: Mineo Yamakawa, Campbell, CA (US); Narayan Sundararajan, San Francisco, CA (US); Andrew Berlin, San Jose, CA (US); Selena Chan, Sunnyvale, CA (US); Xing Su, Cupertino, CA (US); Tae-Woong Koo, Cupertino, CA (US); Lei Sun, Santa Clara, CA (US); Kung-Bin Sung, Seattle, WA (US); Mark Roth, Seattle, WA (US)

Correspondence Address: BLAKELY SOKOLOFF TAYLOR & ZAFMAN, 12400 WILSHIRE BOULEVARD, SEVENTH FLOOR, LOS ANGELES, CA, 90025-1030, US

(21) Appl. No.: 11/325,833

(22) Filed: Dec. 30, 2005

(51) Int. Cl. G01N 33/543; C12M 1/34

(52) U.S. Cl. 436/518; 977/902; 435/287.2

(57) ABSTRACT

Embodiments of the present invention provide methods for determining the degenerate binding capabilities of antibodies. The methods provide information about degenerate binding capabilities without the use of involved procedures. Optionally, a molecule toward which an antibody exhibits degenerate binding ability may be identified through the use of a reporter, such as, a composite organic inorganic nanocluster (COIN). COINs are sensitive SERS (surface enhanced Raman spectroscopy) reporters capable of multiplex analysis of analytes.

### FIELD

Embodiments of the present invention relate generally to the field of Raman spectroscopy, nanoparticle reporters, and the detection of cross-functionality between antibodies and antigens.

### BACKGROUND

Antibodies are naturally-occurring proteinaceous molecules that are a component of the innate and adaptive immune system of vertebrates. In vivo, antibodies defend an organism against infection by binding to viruses and microbial toxins, thereby inactivating them. The binding of antibodies to invading pathogens recruits various types of white blood cells and a system of blood proteins to attack the infectious invaders. In vivo, antibodies are produced in billions of forms. Naturally-occurring antibodies typically have two recognition sites, called antigen binding sites that specifically recognize and bind to an antigenic site on a target invader. A given molecule may present more than one different antigenic site.

Antibodies have found applications as diagnostic agents and therapeutic treatments in humans (such as for auto-immune diseases). Additionally, antibodies have been employed as research tools, such as, for the study of cellular function and the isolation of biomolecules, through for example, immunoprecipitation, immunoblots, immunoassays, cell surface staining. The process of generating and or engineering specific antibodies for specific applications requires tremendous effort. Traditionally the production of an antibody, such as a monoclonal antibody, requires the isolation of an immunogen, immunization of an animal, screening for the antibody of interest, purification, and commercialization which can take years, for example.

The ability to detect and identify trace quantities of analytes has become increasingly important in many scientific disciplines, ranging from part per billion analyses of pollutants in sub-surface water to analysis of treatment drugs and metabolites in blood serum. Among the many analytical techniques that can be used for chemical analyses, surface-enhanced Raman spectroscopy (SERS) has proven to be a sensitive method. A Raman spectrum, similar to an infrared spectrum, consists of a wavelength distribution of bands corresponding to molecular vibrations specific to the sample being analyzed (the analyte). Raman spectroscopy probes vibrational modes of a molecule and the resulting spectrum, similar to an infrared spectrum, is fingerprint-like in nature. As compared to the fluorescent spectrum of a molecule which normally has a single peak exhibiting a half peak width of tens of nanometers to hundreds of nanometers, a Raman spectrum has multiple structure-related peaks with half peak widths as small as a few nanometers.

To obtain a Raman spectrum, typically a beam from a light source, such as a laser, is focused on the sample generating inelastically scattered radiation which is optically collected and directed into a wavelength-dispersive spectrometer. Although Raman scattering is a relatively low probability event, SERS can be used to enhance signal intensity in the resulting vibrational spectrum. Enhancement techniques make it possible to obtain a 10⁶ to 10¹⁴ fold Raman signal enhancement.

### BRIEF DESCRIPTION OF THE FIGURES

FIG. 1 provides a flow chart outlining a method for determining the degenerate binding ability of antibodies.

FIG. 2 provides a diagram of a method for determining the degenerate binding ability of antibodies.

FIG. 3 is a Surface Enhanced Raman Spectroscopy (SERS) spectrum of degenerate binding assays.

FIG. 4 is a SERS spectrum of a negative control without antibodies.

### DETAILED DESCRIPTION OF THE INVENTION

As used herein, the term antibody is used in its broadest sense to include polyclonal and monoclonal antibodies, as well as antigen binding fragments of such antibodies. An antibody useful in a method of the invention, or an antigen binding fragment thereof, is characterized, for example, by having specific binding activity for an epitope of an analyte. The antibody, for example, includes naturally occurring antibodies as well as non-naturally occurring antibodies, including, for example, single chain antibodies, chimeric, bifunctional and humanized antibodies, as well as antigen-binding fragments thereof. Such non-naturally occurring antibodies can be constructed using solid phase peptide synthesis, can be produced recombinantly or can be obtained, for example, by screening combinatorial libraries consisting of variable heavy chains and variable light chains. These and other methods of making, for example, chimeric, humanized, CDR-grafted, single chain, and bifunctional antibodies are well known to those skilled in the art.

The term antigen refers to the molecules that can be recognized (bound) by an antibody. Antigens are most commonly polypeptides or carbohydrates, but they can also be lipids, nucleic acids, or even small molecules like neurotransmitters. A particular antibody molecule can typically only interact with a small region of an antigen and in the case of a polypeptide this is generally about 5-12 amino acids. This region can be continuous or it can be distributed in different regions of a primary structure that are brought together because of the secondary or tertiary structure of the antigen. The region of an antigen that is recognized by an antibody is called an epitope. A particular antigen may have one or more epitotic sites.

The term monoclonal antibody may include an antibody composition having a homogeneous antibody population derived from only one clone of cells, although the scope of the invention is not limited in this respect. In embodiments of the invention, the term monoclonal antibody is not limited to or by the source of the antibody, species, manner in which it is made, isotype, or structure.

As described more fully herein, composite organic inorganic nanoclusters (COINs) are composed of a metal and at least one organic Raman-active compound. Interactions between the metal of the clusters and the Raman-active compound(s) enhance the Raman signal obtained from the Raman-active compound(s) when the nanoparticle is excited by a laser. COINs according to embodiments of the present invention can perform as sensitive reporters for use in analyte detection. Since a large variety of organic Raman-active compounds can be incorporated into the nanoclusters, a set of COINs can be created in which each member of the set has a Raman signature unique to the set. Thus, COINs can also function as sensitive reporters for highly parallel analyte detection. Furthermore, not only are the intrinsic enhanced Raman signatures of the nanoparticles of the present invention sensitive reporters, but sensitivity may also be further enhanced by incorporating thousands of Raman labels into a single nanocluster and or attaching multiple nanoclusters to a single analyte.

It was found that aggregated metal colloids fused at elevated temperature arid that organic Raman labels could be incorporated into the coalescing metal particles. These coalesced metal particles formed stable clusters and produced intrinsically enhanced Raman scattering signals for the incorporated organic label(s). The interaction between the organic Raman label molecules and the metal colloids has mutual benefits. Besides serving as signal sources, the organic molecules induce a metal particle association that is in favor of electromagnetic signal enhancement. Additionally, the internal nanocluster structure provides spaces to hold Raman label molecules, especially in the junctions between the metal particles that make up the cluster. In fact, it is believed that the strongest enhancement is achieved from the organic molecules located in the junctions between the metal particles of the nanoclusters.

The nanoclusters can be prepared using standard metal colloid chemistry. Generally, the nanoclusters are less than 1 μm in size, and are formed by particle growth in the presence of organic compounds. The preparation of such nanoparticles also takes advantage of the ability of metals to adsorb organic compounds. Indeed, since Raman-active organic compounds are adsorbed onto the metal cluster during formation of the metallic colloids, many Raman-active organic compounds can be incorporated into a nanoparticle. Not only can COINs be synthesized with different Raman labels, but COINs may also be created having different mixtures of Raman labels and also different ratios of Raman labels within the mixtures.

Table 1 provides examples of the types of organic compounds that can be used to build COINs. In general, Raman-active organic compound refers to an organic molecule that produces a unique SERS signature in response to excitation by a laser. Typically the Raman-active compound has a molecular weight less than about 500 Daltons.

TABLE 1

| Abbreviation | Name | Structure |
|---|---|---|
| AOH | Acridine Orange Hydrochloride | 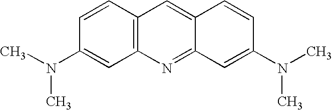 |
| CVA | Cresyl Violate Acetate | 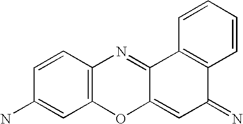 |
| AFN | Acriflavine Neutral | 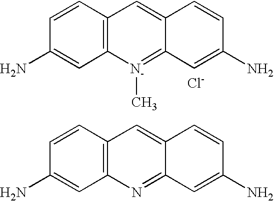 |
| DMB | Dimidium Bromide | 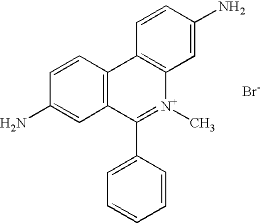 |
| TMP | 5,10,1 5,20-Tetrakis(N-methyl-4- pyridinio)porphyrin Tetra(p- toluenesulfonate) | 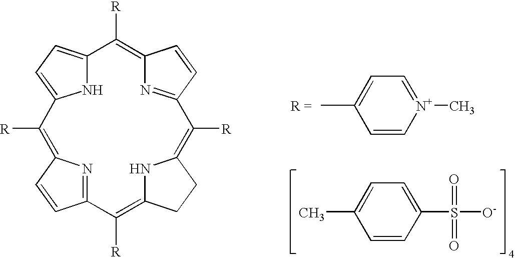 |
| TTP | 5,10,1 5,20-Tetrakis(4- trimethylaminophenyl)porphyrin Tetra(p-toluenesulfonate) | 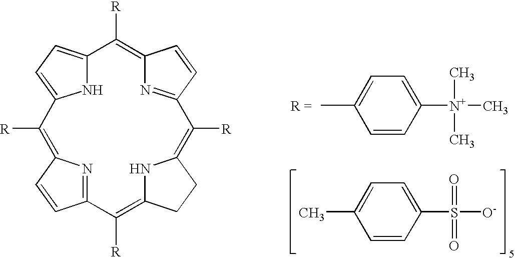 |
| DAA | 3,5-Diaminoacridine Hydrochloride | 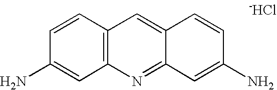 |
| PII | Propidium Iodide (3,8-diamino-5-(3- diethylaminopropyl)-6- phenylphenanthridinium iodide methiodide | 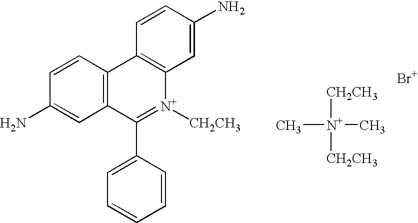 |
| MPI | Trans-4-[4-(dimethylamino)styryl]-1- methylpyridinium iodide | 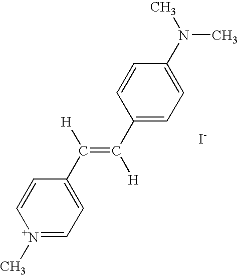 |
| DAB | 4-((4- (dimehtylamino)phenyl)azo)benzoic acid, succinimidyl ester | 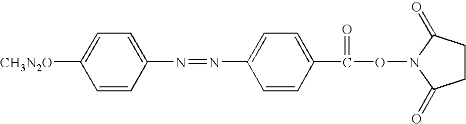 |

In general, COINs can be prepared by causing colloidal metallic nanoparticles to aggregate in the presence of an organic Raman label. The colloidal metal nanoparticles can vary in size, but are chosen to be smaller than the desired size of the resulting COINs. For some applications, for example, in the oven and reflux synthesis methods, silver particles ranging in average diameter from about 3 to about 12 nm were used to form silver COINs and gold nanoparticles ranging from about 13 to about 15 nm were used to make gold COINs. In another application, for example, silver particles having a broad size distribution of about 10 to about 80 nm were used in a cold synthesis method. Additionally, multi-metal nanoparticles may be used, such as, for example, silver nanoparticles having gold cores. In general, for applications using COINs as reporters for analyte detection, the average diameter of the COIN particle should be less than about 200 nm. Typically, in analyte detection applications, COINs will range in average diameter from about 30 to about 200 nm.

Antibody-based probe molecules may be adsorbed to the surface of the COINs or the COINs may be coated before antibody attachment. Typical coatings include coatings such as metal layers, adsorption layers, silica layers, hematite layers, organic layers, and organic thiol-containing layers. Typically, the metal layer is different from the metal used to form the COIN. Additionally, a metal layer can typically be placed underneath any of the other types of layers. Many of the layers, such as the adsorption layers and the organic layers provide additional mechanisms for probe attachment. For instance, layers presenting carboxylic acid functional groups allow the covalent coupling of a biological probe, such as an antibody, through an amine group on the antibody.

For example, COINs can be coated with an adsorbed layer of protein. Suitable proteins include non-enzymatic soluble globular or fibrous proteins. For applications involving molecular detection, the protein should be chosen so that it does not interfere with a detection assay, in other words, the proteins should not also function as competing or interfering probes in a user-defined assay. By non-enzymatic proteins is meant molecules that do not ordinarily function as biological catalysts. Examples of suitable proteins include avidin, streptavidin, bovine serum albumen (BSA), transferrin, insulin, soybean protein, casine, gelatine, and the like, and mixtures thereof. A bovine serum albumen layer affords several potential functional groups, such as, carboxylic acids, amines, and thiols, for further functionalization or probe attachment. Optionally, the protein layer can be cross-linked with EDC, or with glutaraldehyde followed by reduction with sodium borohydride.

In general, probes can be attached to metal-coated COINs through adsorption of the probe to the COIN surface. Alternatively, COINs may be coupled with probes through biotin-avidin coupling. For example, avidin or streptavidin (or an analog thereof) can be adsorbed to the surface of the COIN and a biotin-modified probe contacted with the avidin or streptavidin-modified surface forming a biotin-avidin (or biotin-streptavidin) linkage. Optionally, avidin or streptavidin may be adsorbed in combination with another protein, such as BSA, and/or optionally crosslinked. In addition, for COINs having a functional layer that includes a carboxylic acid or amine functional group, probes having a corresponding amine or carboxylic acid functional group can be attached through water-soluble carbodiimide coupling reagents, such as EDC (1-ethyl-3-(3-dimethyl aminopropyl)carbodiimide), which couples carboxylic acid functional groups with amine groups. Further, functional layers and probes can be provided that possess reactive groups such as, esters, hydroxyl, hydrazide, amide, chloromethyl, aldehyde, epoxy, tosyl, thiol, and the like, which can be joined through the use of coupling reactions commonly used in the art. For example, Aslam, M and Dent, A, Bioconjugation: Protein Coupling Techniques for the Biomedical Sciences, Grove's Dictionaries, Inc., (1998) provides additional methods for coupling biomolecules, such as, for example, thiol maleimide coupling reactions, amine carboxylic acid coupling reactions, amine aldehyde coupling reactions, biotin avidin (and derivatives) coupling reactions, and coupling reactions involving amines and photoactivatable heterobifunctional reagents.

Solid support, support, and substrate refer to a material or group of materials having a rigid or semi-rigid surface or surfaces. In some aspects, at least one surface of the solid support will be substantially flat, although in some aspects it may be desirable to physically separate'synthesis regions for different molecules with, for example, wells, raised regions, pins, etched trenches, or the like. In certain embodiments, the solid support may be porous. Solid substrate may include a bead, plate, tube, filter, particle, or any other suitable material and is not limited to composition, size, shape, or any other physical constraints.

Substrate materials useful in embodiments of the present invention include, for example, silicon, porous silicon, metal-coated surfaces, bio-compatible polymers such as, for example poly(methyl methacrylate) (PMMA) and polydimethylsiloxane (PDMS), glass, SiO₂ (such as, for example, a thermal oxide silicon wafer such as that used by the semiconductor industry), quartz, silicon nitride, functionalized glass, gold, platinum, and aluminum. Functionalized surfaces include for example, amino-functionalized glass, carboxy functionalized glass, and hydroxy functionalized glass. Additionally, a substrate may optionally be coated with one or more layers to provide a surface for molecular attachment or functionalization, increased or decreased reactivity, binding detection, or other specialized application.

Antibodies may be placed on the substrate surface in the form of an array. An array is an intentionally-created collection of molecules housed on a solid support in which the identity or source of a group of molecules is known based on its location on the array. The molecules housed on the array and within a feature of an array can be identical to or different from each other.

Embodiments of the present invention provide the ability to detect cross-functionality between specific antibodies and antigens generally not previously recognized as having binding affinity. Typically, antibodies from a specific species, such as goat, mouse, sheep, rat, rabbit, or hamster, have affinity toward antigens of the same species. In accordance with at least one or more embodiments, antibodies from a non-human species may be used to recognize antigens present in human serum. For example, existing libraries of antibodies can be used to identify the presence or absence of disease, such as cancer, in a human patient serum.

Monoclonal antibodies may be immobilized on to a solid substrate and exposed to human serum for a sufficient time to allow binding to antigens in the human serum. Subsequently, a binding event may be detected by utilizing Surface Enhanced Raman Spectroscopy (SERS) signals without requiring use of a label. In an alternative embodiment, monoclonal antibodies may be immobilized on to a solid substrate, exposed to human serum for a sufficient time to allow binding to antigens in the human serum, and then exposed for a sufficient time to allow binding to an antibody conjugated to COINS for performing a sandwich type assay. In another embodiment, polyclonal antibodies may be immobilized on a solid substrate, exposed to human serum for a sufficient time to allow binding to antigens in the human serum, and then exposed for a sufficient time to allow binding to antigens in the human serum. A binding event may be detected by utilizing SERS signals without requiring utilization of a label. In yet another embodiment, polyclonal antibodies may be immobilized on a solid substrate, exposed to human serum for a sufficient time to allow binding to antigens in the human serum, and then exposed for a sufficient time to allow binding to an antibody conjugated to COINS for performing a sandwich type assay. The resulting data may then be analyzed to compare one or more results between human serum from cancer patients and non-cancerous patients, and to determine information therefrom.

Numerous antibodies suitable for utilization in accordance with the present technology are available, both commercially available or currently being researched. For example, monoclonal antibodies are available from the Developmental Studies Hybridoma Bank (http://www.uiowa.edu/˜dshbwww/).

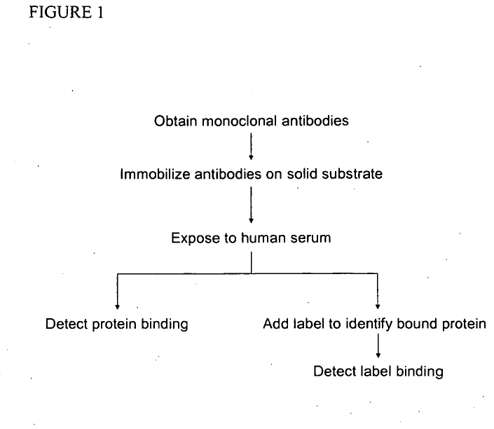

FIG. 1 provides a flow chart outlining a method for determining the degenerate binding ability of antibodies. To test the degenerate binding ability of the monoclonal antibody, the first step is to obtain and immobilize the antibodies on a solid substrate. Once the antibody is immobilized on the substrate, human serum is added and if the antibody is degenerate, it will bind to proteins in the human serum. The bound protein is detected using surface enhanced Raman scattering (SERS). To identify the bound protein, a label can be introduced, such as COIN, which is a metal nanoparticle aggregate that generates a unique SERS signal. The COIN may be conjugated with a detection antibody that recognizes the bound protein. The bound protein with COIN attached is detected using surface enhanced Raman scattering (SERS).

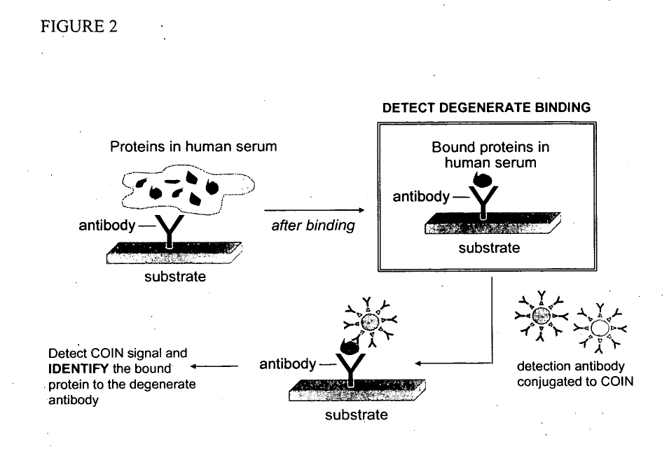

FIG. 2 show a diagram of degenerate binding in accordance with embodiments of the invention. An antibody is immobilized onto a solid substrate. Human serum is then added. If the antibody is degenerate it will bind to protein or other molecules in the human serum. The remaining serum is then washed from the surface of the substrate. The bound antigen is detected using surface enhanced Raman scattering (SERS). In FIG. 2, to identify the bound protein, a label can be introduced, such as, for exmaple, COIN, which is a metal nanoparticle aggregate that generates a unique SERS signal, or a quantum dot. The COIN particle attaches to the bound antigen through, for example, detection antibody that recognizes a second epitope of the bound antigen. The bound antigen with COIN attached is detected using surface enhanced Raman scattering (SERS).

SERS of the substrate-attached antibody antigen complex can be performed for example, by depositing a solution of metal nanoparticles (such as, for example silver nanoparticles) on the surface of the substrate. The silver nanoparticle solution may optionally contain a signal enhancer, such as LiCl. The term metal or metal nanoparticles may in general refer to and may encompass any metallic structure which may include any structure made wholly, partially, in mixture, or in layers of metal, and which may include rough metal, metal colloids, metal nanoparticles, metal films, and metal coatings, although the scope of the invention is not limited in this respect. Additionally, metal-coated substrates, such as metal-coated silicon or metal-coated porous silicon can function as SERS substrates.

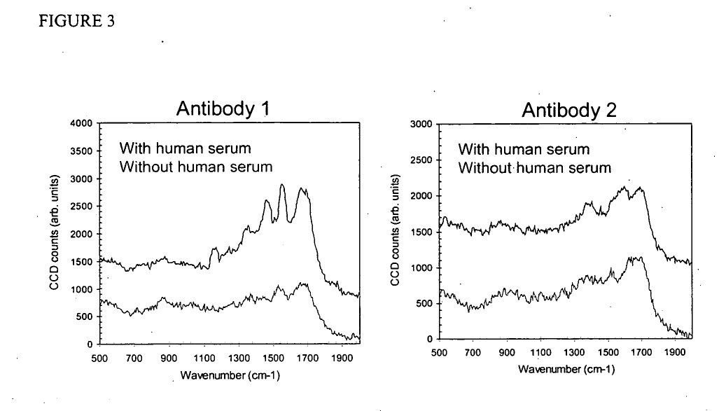

FIG. 3 shows a SERS spectrum from two different monoclonal antibodies, antibody 1 and antibody 2. For antibody 1, unique spectral features are observed when proteins in human serum bind to the antibody as compared to spectral features without human serum. Therefore, antibody 1 exhibits degenerate binding ability for proteins in human serum. However, for antibody 2, no unique spectral features are observed when human serum is reacted as compared to spectral features without human serum, indicating that antibody 2 does not have degenerate binding ability for proteins in human serum.

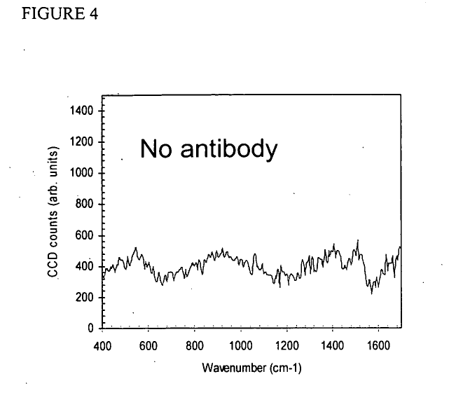

To ensure that the SERS signal was not due to non-specific binding of the proteins in human serum to the substrate, experiments were conducted without the presence of antibodies. FIG. 4 shows that the SERS spectrum is relatively flat and does not contain the strong peaks observed in antibody 1. This may serve as a reference to determine whether non-specific binding of proteins in human serum generate a SERS signal.

We claim:

1. A method of investigating antibody reactivity comprising,
immobilizing one or more antibodies onto a surface of a solid substrate;
performing surface enhanced Raman spectroscopy (SERS) on the substrate surface;
contacting the immobilized one or more antibodies with a solution containing a one or more molecules in a manner that allows specific binding of one or more molecules to one or more immobilized antibodies;
removing any unbound molecules from the surface of the substrate;
performing surface enhanced Raman spectroscopy (SERS) on the substrate surface a second time; and
determining the presence or absence of bound molecules on the substrate surface through a comparison of a first and second surface enhanced Raman spectroscopy (SERS) spectrum.

2. The method of claim 1, wherein the antibody is a monoclonal or a polyclonal antibody.

3. The method of claim 1, wherein the antibody is a monoclonal antibody derived from a human.

4. The method of claim 1, wherein the antibody is a monoclonal antibody is from a non-human species.

5. The method of claim 1, wherein the solution of molecules is serum from a mammal.

6. The method of claim 1, wherein a plurality of different antibodies are attached to the substrate and comprise and array.

7. The method of claim 1, wherein the solid substrate is comprised of silicon, porous silicon, a silver-coated surface, a gold-coated surface, poly(methyl methacrylate) (PMMA), polydimethylsiloxane (PDMS), glass, SiO₂, quartz, silicon nitride, functionalized glass, gold, silver, platinum, or aluminum.

8. The method of claim 1, also including contacting a solution containing a reporter molecule having an antibody specific for an epitope of a molecule in the solution with the substrate surface after contacting the immobilized one or more antibodies with a solution containing a one or more molecules in a manner that allows specific binding of one or more molecules to one or more immobilized antibodies, in a manner that allows specific binding of the antibody attached to the reporter to a molecule, removing any unbound reporters, and determining the presence or absence of the reporters on the substrate surface.

9. The method of claim 8, wherein the reporter is a composite organic inorganic nanocluster (COIN) and determining the presence of the reporter occurs by detection of a Raman signal.

10. The method of claim 1 wherein performing surface enhanced Raman spectroscopy (SERS) on the substrate surface consists of contacting an antibody or an antibody antigen complex with a silver or gold surface and obtaining an enhanced Raman spectrum from the antibody or the antibody complex.

11. A method of investigating antibody reactivity comprising,
immobilizing one or more antibodies on a surface of a solid substrate;
contacting the immobilized one or more antibodies with a solution containing a one or more molecules in a manner that allows specific binding of one or more molecules to one or more immobilized antibodies;
removing any unbound molecules from the surface of the substrate;
contacting a solution containing a reporter molecule having an antibody specific for an epitope of a molecule in the solution with the substrate surface in a manner that allows specific binding of the antibody attached to the reporter to a molecule;
removing any unbound reporters; and
determining the presence or absence of the reporters on the substrate surface.

12. The method of claim 11, wherein the antibody is a monoclonal or a polyclonal antibody.

13. The method of claim 11, wherein the antibody is a monoclonal antibody derived from a human.

14. The method of claim 11, wherein the antibody is a monoclonal antibody is from a non-human species.

15. The method of claim 11, wherein the solution of molecules is serum from a mammal.

16. The method of claim 11, wherein a plurality of different antibodies are attached to the substrate and comprise and array.

17. The method of claim 11, wherein the solid substrate is comprised of silicon, porous silicon, a silver-coated surface, a gold-coated surface, poly(methyl methacrylate) (PMMA), polydimethylsiloxane (PDMS), glass, SiO₂, quartz, silicon nitride, functionalized glass, gold, silver, platinum, or aluminum.

18. The method of claim 11, wherein the reporter is a composite organic inorganic nanocluster (COIN) and determining the presence of the reporter occurs by detection of a Raman signal.
